(19) United States
(12) Patent Application Publication
(10) Pub. No.: US 2022/0333168 A1
(43) Pub. Date: Oct. 20, 2022

(54) METHOD FOR IDENTIFYING AND QUANTIFYING ORGANIC AND BIOCHEMICAL SUBSTANCES

(71) Applicant: Roswell Biotechnologies, Inc., San Diego, CA (US)

(72) Inventors: Doris Steinmüller-Nethl, Aldrans (AT); Anton Kock, Vienna (AT); Detlef Steinmuller, Aldrans (AT); Kriemhilt Roppert, Kirchstetten (AT)

(73) Assignee: Roswell Biotechnologies, Inc., San Diego, CA (US)

(21) Appl. No.: 17/699,980

(22) Filed: Mar. 21, 2022

Related U.S. Application Data
(63) Continuation of application No. 16/696,604, filed on Nov. 26, 2019.
Application No. 16/696,604 is a continuation of application No. 12/667,583, filed on Mar. 5, 2010, now abandoned.

(30) Foreign Application Priority Data
Jul. 4, 2007 (AT) A 1033/2007

(51) Int. Cl. C12Q 1/6825; G01N 33/543

(52) U.S. Cl. CPC C12Q 1/6825; G01N 33/5438; C08G 71/02

(57) ABSTRACT

A method for identifying and quantifying organic or biochemical substances in a fluid medium using a nanogap sensor is disclosed. A nanogap sensor with two electrodes of different materials is used, a respective probe molecule is bonded to each electrode and the free remainder of the probe molecules have at least one bondable group with specificity to a substance or analyte. The analyte has at least two binding sites and passes selectively out of the fluid medium, binds to the free ends of the probe molecules to form a bridge, modifying the impedance between the electrodes.

### CROSS-REFERENCE TO RELATED APPLICATIONS

This application is a continuation and claims priority from U.S. patent application Ser. No. 16/696,604 filed Nov. 26, 2019 entitled “Method for Identifying and Quantifying Organic and Biochemical Substances,” which is a continuation and claims priority from U.S. patent application Ser. No. 12/667,583 filed Mar. 5, 2010 and entitled “Method for Identifying and Quantifying Organic and Biochemical Substances.” The '583 application is the National Stage Entry under § 371 of International Application Serial No. PCT/AT2008/000242, filed Jul. 4, 2008 and entitled “Method for Identifying and Quantifying Organic and Biochemical Substances.” The '242 PCT application claims priority to and the benefit of Austrian Provisional Patent Application Serial No. A 1033/2007, filed Jul. 4, 2007 and entitled “Method for Identifying and Quantifying Organic and Biochemical Substances,” the disclosures of which are incorporated herein in their entireties.

### FIELD

The present invention relates to a new method for identifying substances and more particularly to a method for identifying particular molecules, molecule sequences, molecule parts, or the like, and for determining their quantity or concentration in a fluid.

### BACKGROUND

The identification of nucleic acids has many applications, which e.g., include the identification of pathological organisms, genetic tests and forensic expertises. In the automation of simultaneous screening of thousands of characteristic nucleic acid sequences, considerable progress has been achieved: in gene chip or micro-array technology, many different DNA samples are exactly positioned on glass or silicon chips and immobilized in doing so. The sample to be investigated is contacted with the chip, and only with complementary nucleic acids being present in the sample, it hybridizes with the probe DNA on the chip. Fluorescence detection is subsequently used to detect the resulting double-strand nucleic acid products. The advantage of this system lies in the fact that hundreds to thousands of sequences can be examined by automatic systems as well as that respective systems are commercially available.

Hybridization detection using fluorescence is therefore per se a powerful method for the specific detection of nucleic acids. But still, in order to obtain a detectable and reliable signal with this system, for detection, first the target molecule in the sample has to be selectively proliferated using PCR; additionally, marking with fluorescence markers is required. Consequently, for evaluation, this technology also requires a system, which can detect fluorescence. For these reasons, this established system is very complex, and thus simpler, more direct methods are desired and required.

The suggestion for the solution of this problem according to the invention presented here relates to the use of electrical nano-biosensors for the detection of biological molecules, preferably of nucleic acids.

Why Electrical Nanogap Sensors?

Biosensors are sensors, on the surface of which biocomponents, i.e. probe molecules, are immobilized, which again interact as sensor elements with the analyte and can transmit their reaction to a transducer. Thus, the actual detection takes place directly on the surface of the electrodes. Impedimental in this is, up to a certain the degree, the electrical double-layer capacity, i.e. the electrode polarization, which is determined by the accumulation of ions in the proximity of the electrode surface. Thus, it becomes difficult to measure the properties of biological molecules, which in a biosensor, according to the definition, are immobilized on the sensor surface; thus, this also has a negative influence on the detection of the analyte, above all at lower frequencies.

Small nanogap sizes or dimensions, respectively, on the other hand minimize polarization effects of the electrodes, namely depending on the frequency. If the nanogap is chosen smaller than the thickness of the electrical double layer, the dependency of the nanogap capacity from the ion strength disappears. This is particularly important, when during the course of the detection process there is a modification of the ion strength, e.g. due to washing processes.

Types of Nanogap Sensors

Nanogap sensors published so far are either based on the measurement of dielectric effects in order to distinguish single-strand or double-strand DNA in solution from one another, or use DNA strands to create a more or less conductive connection between individual electrodes.

For dielectric sensors, modifications of capacity or other impedance-based data are chosen as indicators for the existence of the target molecule or its conformation.

According to another approach, two electrodes are, for example, interlinked by nucleic acids. An increase of conductivity between these two electrodes is measured. Consequently, electrically conductive biological molecules are required. The conductivity may be significantly increased by metallization of the DNA strands (Braun et al.).

### SUMMARY

With a completely new access, the new approach suggested here has the advantages of these two approaches mentioned. Cross-linking reactions with alternating current measurements are used. With a certain arrangement, the efficiency of the cross-linking reaction is increased, and thus the detection limit significantly decreased.

The present invention relates to a new method for identifying substances, in particular molecules, molecule sequences, molecule parts or the like, and for determining their quantity or concentration, respectively, in a fluid, i.e. liquid or even gaseous medium, using a nanogap sensor that comprises at least two electrodes, according to the recitations of additional aspect 1 below, which has the characteristics stated in the characterizing part of this additional aspect.

### BRIEF DESCRIPTION OF THE DRAWING FIGURES

FIG. 1a schematically shows a novel arrangement of electrodes 1 and 2 of a nanogap sensor 100;

FIG. 1b shows formation of a bridge Bm between electrodes 1 and 2;

FIG. 2 shows an inverse process beginning with an existing bridge Bm;

FIG. 3 shows a similar process to FIG. 2, wherein molecules E,7 present in the fluid medium Mf bind to only one of the two peripheral binding sites d63,d64 of the auxiliary molecule D,6, namely d64; and

FIG. 4 shows an existing bridge formed with probe molecules A,3 and B,4, sensor-bound to two electrodes 1 and 2, destroyed by enzyme E8 in three different ways.

### DETAILED DESCRIPTION

A nanogap, which is defined by two electrodes of different materials, is bridged due to the bond of the analyte or analyte molecule, respectively, or an auxiliary molecule to two different probes or probe molecules, respectively. Various probes are respectively immobilized on various electrodes, and on each of the electrodes, only one type of probe is present. Each analyte or auxiliary molecule, respectively, has two different exposed binding sites for the two affinity binding sites of the two different probes, which are sensor-bound to the electrodes of different materials and thus immobilized there. The detection of this link takes place using the alternating current analysis between the electrodes before or after, respectively, the bonding event or even with continuous temporal recording, corresponding to online recording in real-time.

Background of the approach used according to the invention—More efficient linking

According to this new approach, the electrodes are to be linked with one another, e.g. by DNA strands, and thus detection of the analyte is to be affected. For efficient linkage of the electrodes, it is important that there is no competitive reaction. In nanogap sensor configurations published so far, this mostly was not the case: only a small share of the possible DNA strands bridged the nanogap, most of them reacted in other reactions already and e.g. formed “inner electrode loops.”

A clear improvement of the reaction responsible for the measuring signal represents a substantial component of the present invention.

Considering, e.g., patent application US 2006/0019273 A1, and especially FIG. 12 there, it can be noticed that due to immobilized scavenger molecules, a competitive reaction is possible by loop formation, which, however, is not mentioned in the US patent application: both ends of the nucleic acid sequence to be detected can bind to the same electrode, which is why there is no bridging of the individual different electrodes and thus also no substantial contribution to the detection signal. Due to the steric circumstances it is evident, that such loop formation is even preferred compared to the bridging of the nanogap, and consequently the detectable events do not correspond to the principally occurred binding events.

In Hashioka et al., the DNA is even only attached at one electrode, thus, too, the bridging of the nanogap by the DNA surely is little effective.

Patent application US 2002/0172963 A1 mentions the importance of not admitting any contact of the DNA with the support wafer, onto which the nanogap electrodes are applied. This represents a step to that effect that the efficiency of the binding events is to be increased, however, it does not state anything about the prevention of other possible side reactions. Thoughts about an optimal orientation of the DNA to be measured are also not present there.

Another extremely important characteristic of sensors for the analytic nucleic acid chemistry is selectivity: the detection of point mutations, i.e. individual modified bases, is gaining more and more significance. Methods as to how point mutations can be detected, are sufficiently described per se in the literature, e.g. in Sambrook et al., Molecular Cloning. Homologous nucleotide sequences can principally be detected by selective hybridization; for an increase of selectivity, so-called stringent conditions, like low ion strengths and increased temperature, are used.

Another concept consists in the increase of selectivity by the synchronous use of two probes; this can be found, e.g., in sandwich hybridizations and in real-time PCR. For that, two different probes must be bound at the same time in order to be able to detect the binding event. Bridging reactions per se are predestined for such probe systems, but still e.g. Hashioka et al. and US 2006/0019273 A1 renounce them.

From these examples emanates the fact that the efficiency of bridging or of competing side reactions, respectively, represents an important role for the lowering of the detection limit in compliance with the demanded selectivity for the usability of the nanogap sensors.

In terms of the present invention it is suggested to selectively occupy those two electrodes, which are to be bridged, with respectively only one type of probe molecule, so that “inner electrode loops” are not possible. Thus, all binding events taking place are forced to bridge the nanogap and thus to contribute to the detector signal.

Immobilization—The Problem

When using nano-scaled electrodes, however, the methods for oriented selective immobilization e.g. used in micro-array technology cannot be applied, since classical spot sizes have a diameter of about 100 μm and a distance from one another of 100 to 400 μm, i.e. the wrong magnitude. Likewise, for nano-scaled electrodes, the limits of classical lithography have already been reached. Consequently, another approach is required. Additionally, increased temperatures for achieving the necessary selectivity furthermore also include the necessity to ensure stable bonds of the biomolecules to the sensor surface: the thiol-gold bond commonly used for electrode systems is only a quasi-covalent, but not a real covalent bond. This clearly emanates from the binding energies involved. Thus, at temperatures normally necessary for stringent conditions, the gold-thiol bond already becomes thermally unstable.

Due to the sensor concept of nanogap bridging, however, even a loss of only a small part of the DNA coupled via a thiol bond means an enormous drift, which will cover the signal of the occurred hybridization.

For this reason, the popular thiol-gold system may be suitable for detection in case of “stronger deviating” sequences, however, not in case of point mutations. In this case, other systems with a stronger bond to the electrodes must be found and used. Additionally, for a future product, it must be possible to perform the production chain within the established tracks of semiconductor technology.

Likewise, it is clear that for electrode systems, which have nm dimensions, possibly required intermediate layers between the electrodes and the biocomponent must be as thin as possible, i.e. in the nm range or, in the optimal case, there is no necessity for such intermediate layers at all. Occasionally required intermediate layers, however, must be sufficiently well defined, which cannot be achieved with the thiols in reality (hardly controllable multi-layers instead of mono-layers). Thus, a use of longer-chain thiols, which principally are more temperature-stable and thus possibly could be used, likewise is not possible. Thus, in reality, only direct connections to the sensor surface come into consideration.

Immobilization-Solution of the Problem

The selective immobilization in the nano range is efficiently achieved according to the present invention by the fact that the two electrodes defining the nanogap are formed from different materials right from the beginning, since different materials also involve different chemical and physical properties. Chemical reactions used for binding biomolecules at their different surfaces may be designed that way that selectively only a certain one of the different material surfaces can be linked with a certain biomolecule. Thus, in a simple manner it is possible to selectively place probe molecules on certain, small, also nano-scaled areas.

Approaches for selective immobilization published so far did not mention the effective approach as provided according to the invention, i.e. to make use of asymmetrical electrode properties.

Patent application US 2002/0022223 A1 though mentions a separate immobilization on the electrodes, but does not provide a more detailed description of an actually possible execution. The methods mentioned in this document are not practicable for localized immobilization in the nm magnitude. There, electrostatical and/or chemical differences for selective immobilization are contemplated for the minimization of the immobilization on the support material of the electrodes only—but not on the electrodes themselves.

Patent application US 2004/012161 A1 likewise mentions the importance of efficient linking of the individual electrodes via selective immobilization of the individual probes. This takes place via a complex process, which uses nickel electrodes and gold electroplating with poisonous cyanide ions, since, as is appropriately noted in this patent application, mechanical placing of smallest quantities in the nm range is no longer possible. All electrodes, however, principally consist of the respectively same material. These approaches known so far though principally fulfill their target, but they are not accessible for cheap mass production.

In another context, patent application US 2002/0172963 A1 per se shows the idea, not to use immobilization on the electrode substrate and to achieve a selective immobilization via electrostatic effects and detours. This method, however, is unnecessarily complicated and thus likewise not accessible for mass fabrication.

Another published patent application, namely US 2002/0172963 A1, primarily aims at a surface extension by electrically addressable nano-tubes. Selective immobilization is achieved via positive and negative charges as well as gold particles. Thus, again, selective immobilization is not achieved via intrinsic material properties. Additionally, this approach still includes the polarization effects, since this is not a nanogap structure; for the manufacture of these sensors, expensive electron beam lithography is additionally required.

Simple manufacture according to the invention

There is the requirement, that the nano-electrodes can be manufactured in a simple manner.

The required gap widths are roughly determined by the size or length, respectively, of PCR products or other detection-relevant molecules, respectively, and typically lie within the magnitude of about 50 nm. “Conventional” nano-electrodes require e-beam lithography for their manufacture; the costs resulting from that, however, make the product uninteresting for the existing market.

The integration of molecular biology with nano-electronics requires surfaces, which are stable upon contact with biological molecules as well as compatible with the fabrication methods of microelectronics. Additionally, thermally stable bonds of the probe molecules with the sensor surface are required.

Diamond surfaces may be well functionalized with biomolecules. Diamond is biocompatible, chemically extremely stable, has an electro-chemical potential window of 4 V and is absolutely compatible with semiconductor technology. Nano-crystalline diamond layers are deposited onto silicon wafers in order to ensure the requirements for the practice-oriented fabrication and commercialization of the components, since here the well-established, CMOS-compatible processes have advantages. This approach also ensures that established strategies for cost reduction may be applied at a later stage of the new project.

So far, lateral nanogaps with electrodes, which are apart from one another by only a few ten nm, can only be produced with complex electron beam lithography. The reproducibility of these lateral nanogaps, however, is problematic. In order to achieve high sensitivity at low manufacturing costs for DNA chips, metal nanogap electrodes are suggested (Hashioka), but the current approaches require complicated techniques, as for example electron beam lithography (Hwang).

There were reports about alternative nano-fabrication techniques using various methods for manufacturing nanogaps usable for DNA chips at lower costs (Hashioka). These include electro-deposition (Qing et al.), electro-migration (Iqbal), electro-chemical methods (He et al., Liu et al., Chen et al.) and fracture techniques (Reed et al.; Reichert et al.). All these methods, however, have highly limited possibilities for application due to compatibility problems with current high throughput methods in the semiconductor industry.

For the purpose intended according to the invention, the electrodes themselves must have a conductivity, which clearly lies above that of classical undoped semiconductors. Consequently, metals as well as highly doped or highly dopable semiconductor materials, respectively, come into consideration. Non-limiting examples for that are Si- and C-based materials like silicon, diamond or diverse graphite modifications.

Another possibility to realize nanogap sensors relates to layer systems. Layer thicknesses are also reproducible in the nm range and easy to manufacture. If, for example, the middle, i.e. the second layer is etched from a three-layer system, then the width of the gap is exclusively determined by the thickness of the former second layer. This approach is thus absolutely reproducible. The final structuring of the component may be performed using standard lithography. Complicated and expensive electron beam lithography is thus not necessary for the final manufacture of the nanogap element.

Measurement-Problems and Approaches to Improvement

The approaches for bridging nano-electrodes published so far have in common, that conductivity of the DNA is assumed. Especially in respect of DNA, however, there are quite contradictory data in the literature on the conductive or isolating properties. This assumes more complex, currently still device- or application-dependent connections of an unknown kind, which have to be considered.

US 2002/0172963 A1, for example, refers to biological molecules, which are capable of electrical conductivity, and for that states nucleic acids like DNA or RNA. Especially for these, however, the results of the electrical characteristics are rather controversial in the literature. Those are observed in more detail in US 2002/0172963 A1, especially their linker dependencies, and optimized. As a substantial result, no conductivity contribution has to be expected from single-strand DNA, but very well from double-strand DNA. Considering, however, the base lengths of typical PCR fragments, then not only the two probe molecules, which determine the sequence to be detected, are required, but also a “gap filler” or “helper oligo-nucleotide,” see FIG. 8 there; this, however, is not directly mentioned otherwise in the US-A1 stated. This, however, contributes to the complexity of the assay and decreases the efficiency of the detection reaction in any case.

It is obvious, that systems carefully balanced to such an extent are inflexible and adaptations for new situations, like e.g. the modification of the target molecule, may be possible with considerable effort only.

Consequently, it is substantially more productive, as provided according to the present invention, to aim at the electrical measurement of less restricted characteristics: the option to characterize/detect the bridging of sensors via much more sensitive and flexible alternating current measurements instead of direct current curves has not been perceived in the literature so far: in this case, isolating instead of conductive characteristics of analytes are no obstacle anymore for a successful reaction. Alternating current measurements additionally offer the advantage that for the measurement, no or only a very small current must flow. Thus, the biomolecules are not influenced in their behavior by the measurement, and an interference-free online observation of the results is possible.

This, e.g., is not possible with the voltages in the volt range stated in patent application US 2002/0172963 A1 in [0082], which cause irreversible reactions in biomolecules. This prevents a possible observation of biointeractions in real-time. Bridging the gap between the electrodes, the analyte or the analyte or auxiliary molecule, respectively, is also optimally presented and oriented for detection.

Approach, Detection Sequence

### DETAILED DESCRIPTION

1. Sensor fabrication

2. Immobilization; possibility helper oligo-nucleotide; sequence selection

3. Sample preparation, PCR; occasional denaturation of the nucleic acid, as long as this is present as a double-strand

4. Measurement before/during/after; washing; temperature

5. Chip PCR

ad 1: Sensor Fabrication

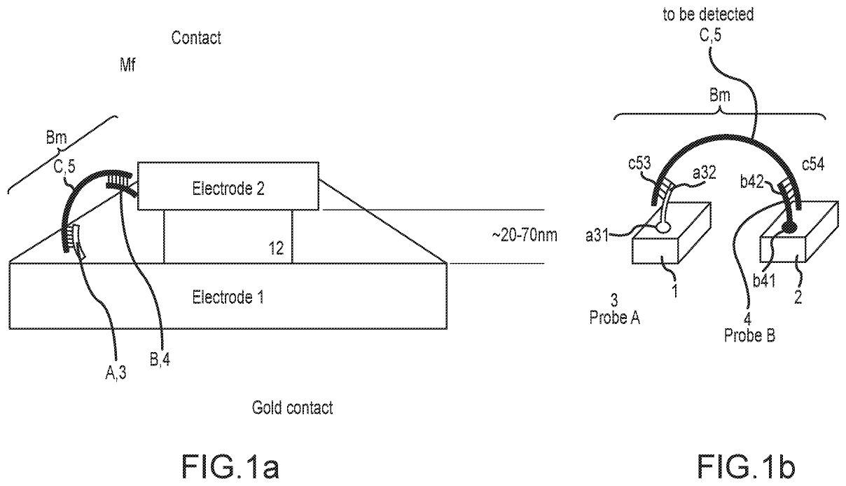

The nanosensor suggested according to the invention is schematically shown in FIG. 1a.

The material combination shown shall only serve as an example and only demonstrates one of the possible variants for execution. For the purpose of clarity, only a single electrode link is represented as a section. However, it is evident that several of these links, too, may be unified in a connected or unconnected form on a chip (“array”).

For manufacture, a n+-doped silicon wafer is thermally oxidined. The thickness of the SiO₂ layer applied this way lies within the magnitude of a few 10 nm. Ultimately, this determines the width of the nanogap.

As the next step, via CVD processes, these wafers are coated with a thin diamond layer with a thickness of 50 to 200 nm. Metal contacts, for example gold, are applied onto the diamond layer using photo-lithography and lift-off processes, in order to guarantee good ohmic contact. These serve as points of contact to the electronic detection and evaluation unit. In the next step, the diamond layer is structured with suitable ion etching techniques. Ultimately, the SiO₂ layer is then wet-chemically undercut or completely etched off, in order to expose the nanogap.

ad 2: Immobilization

Selective and highly precise immobilization is ensured by using various materials for the two electrodes, which e.g. consist of diamond and silicon. This is schematically shown in FIG. 1. Various materials also mean various chemical properties on the surfaces: in combination with the use of selective reactions, covalent bonds only result on certain surfaces. Thus, a localized chemistry at the different electrodes and a resolution into nm regimes becomes possible, in order to force e.g. DNA fragments to bridge the nanogap.

As an—by no means limiting—example, a diamond-silicon nanogap sensor is described in more detail: nitrophenyl groups can be electrochemically immobilized on the diamond surface. These are then converted into aminophenyl groups, and using a crosslinker, like PDITC (chemical name: phenylene diisothiocyanate), commercially available amino-oligos are covalently bound to this surface.

With diamond, however, not only the possibility is given to tailor the morphology and the electrical properties, like isolator behavior, p-conductivity, and semi-metallic behavior; the surface termination, too, can be designed flexibly. For example, hydrogen, oxygen, fluorine and nitrogen terminations are possible. This also enables the application of other chemical and not only electrochemical approaches for the selective immobilization in the nm scale.

As the next step, the other probe molecule can be selectively immobilized on the silicon surface, since the diamond surface has already been blocked off with oligo-nucleotides. Our own work has shown (poster at the Bioelectrochemistry 2005 of Roppert et al. as well as not yet published data), that it is possible to immobilize DNA directly on silicon, without having to use an intermediate silane layer. Once the component has nanoscaled dimensions, which have to be exactly adjusted in size, a not 100% exact intermediate layer between sensor and biomolecule would highly affect the functions of the sensor with the highest probability.

The two different probe molecules are thus selectively applied to the electrodes of different materials, which have a distance from one another, which is determined by the gap. Due to the sequence selection and the chosen conditions for detection, the probes cannot interact with one another; therefore, the aspect described in US 2002/0022223 A1 and US 2005/0287589 A1, that the probes must not touch one another distance-related, is in no way relevant for the invention.

ad 3: Sample Preparation

Isolation, sample preparation and possible purification of the nucleic acids, peptides, proteins or further analytes take place according to the known state of the art methods. The molecules to be detected may also be selectively or non-selectively enriched or proliferated, respectively, before the analysis.

Especially in the case of nucleic acids, proliferation of DNA or “transcription” of RNA into cDNA with simultaneous proliferation may be required.

Upon presence of a double-strand nucleic acid, possibly dematuration of the nucleic acid, e.g. by heat or alkali influence, respectively, must take place for detection.

Special significance, however, has the use as an RNA sensor. Detection of e.g. microorganisms via RNA detection may principally achieve higher sensitivity than such one via DNA, since rRNA molecules are present in higher numbers than the DNA detecting them. Thus, direct detection of nucleic acids can be achieved relatively easily without previous proliferation. This is an advantageous difference to cDNA micro-arrays. For the verification of RNA viruses, like e.g. influenza, too, this is relevant to a high extent.

ad 4: Measurement Before/During/After

In principle, the component is first prepared for the measurement by connecting the contacts with a respective measuring device. The electrode areas are equilibrated with detection buffer without analyte or analyte molecules, respectively. Now, a first measurement of the component takes place under the conditions of the detection reaction. Following determination and possibly stabilization of the initial value only, the analyte or analyte molecules, respectively, are added. The change compared to the initial value can be measured continuously or also following a certain period of time only.

Washing processes and other methods common in biological analytics, like blocking off of non-specific binding sites or temperature increase, may be integrated into this process.

Alternative, common methods currently not used in the industry yet, however, shall not be excluded by that. US 2002/0022223 A1 mentions, e.g., the possibility of using non-aqueous buffers with low electrical conductivity.

Individual sensors, which again may consist of several belts themselves, may be combined with the same or different probes or probe molecules, respectively, into a so-called array on a chip. This arrangement is then especially suitable to detect several to a high number of different components in a single sample, to obtain a representative cross-section over one sample or for diverse control sequences, which e.g. may serve to detect point mutations or carry-over contaminations, comp. e.g. US 2005/0287589 A1.

All these approaches may be integrated with and/or into respective microfluidics, in order to ensure a respective liquid supply under controlled conditions.

Likewise, a reverse approach is possible. For that, an existing bridge is destroyed by the detection event. This is achieved by the fact that the bridging molecule as an “auxiliary molecule” has a higher affinity to the analyte than to the probe molecules, which attach it to the sensor.

In the case of nucleic acids, this may e.g. be achieved by introducing point mutations into the bridge molecule, and for proteins, by not exactly fitting/non-specific antibodies.

Important in this connection are also ligand displacement assays (LDAs). Here, an already bound analyte analogue, which may also be identical with the analyte in terms of structure, may be displaced by the “real” analyte. Therefore, in case of a positive sample, analyte and analyte analogue are in an equilibrium GG with one another. With a more exact optimization of the test, this equilibrium can be shifted towards the bond of “real” analytes. Thus, e.g. an antibody or the like drifts off from the sensor surface, which then causes a signal modification in the solution or also on the sensor surface, respectively. Thus, this is a special case of a competitive test.

With more complex approaches, a drifting off of larger molecule clusters may be caused by the binding or drifting off of an analyte (analogue), which again may drastically increase the signal yield. Thus, a “pre-bound” situation with an analyte (analogue), which may be further conjugated, is present, which is displaced by the analyte, whereby a substantially higher signal modification is caused than a small analyte could trigger itself.

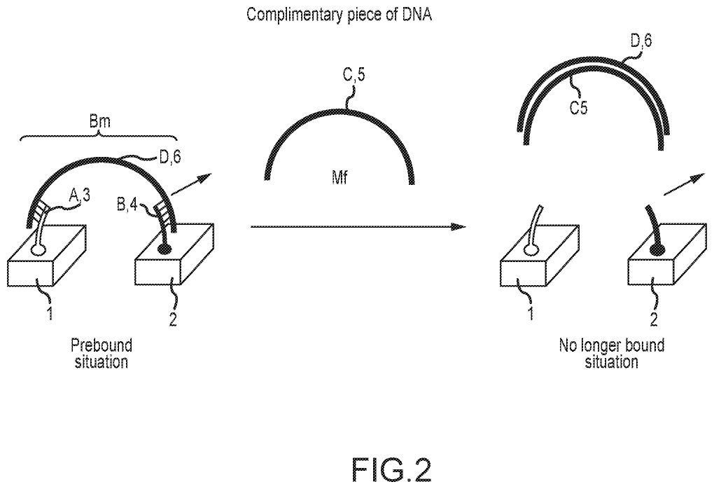

Concrete cases are shown in FIGS. 2 to 4, which will be dealt with later on, and are there explained in more detail.

In all cases, M-DNA techniques may help improving the signal difference between bridging and non-bridging.

Likewise, the use of so-called “helper oligo-nucleotides”, which result in a continuous double-strand situation, can be implemented in all cases.

As measuring methods, above all impedance methods are being considered. Various frequencies may be used, or also entire spectra may be traced. These may be provided with a DC offset, or there may be measurements with OCP (open circuit potential) or with floating potential methods. Likewise, an external reference electrode may be used or such one may be integrated at the chip. Four-point measurements may likewise be used. These procedures are measuring methods corresponding to the state of the art, however, by no means exclude other methods.

Ad 5: Chip PCR

This arrangement is also suitable for on-chip PCR. In principle, two arrangements are possible here: either the selectively immobilized primers are linked with one another analogue to a “normal” PCR reaction using polymerase chain reaction, or the approach follows the TaqMan system: a primer is immobilized at an electrode and the “sample” is immobilized at the other electrode. The second primer is free in solution. If now a PCR product is synthesized, first, during the annealing step, the gap is bridged, and then, during the subsequent polymerization/extension, the bridge bond between primer 1 and the “sample” is hydrolyzed again by the 5′-3′ exonuclease activity of the AmpliTaq DNA polymerase. If, on the other hand, no product is formed, there is no bridging of the nanogap during the reaction, and thus no modification of the signal.

Additional aspects 2 to 6 set forth below relate to various preferred embodiments of the present invention; in particular, additional aspects 2 and 3 set forth below relate to various types of approaches for the resolution of an initially existing bridge formed with probe molecules and analyte molecule or analyte analogue molecule between the electrodes of different material, and additional aspects 4 to 6 set forth below relate to favorable embodiments of the nanogap sensors essential for the invention.

Finally, additional aspects 7 to 10 set forth below relate to various types of use of the new analysis technology according to the invention in the nm range.

The invention is set forth in more detail on the basis of the figures.

FIG. 1a schematically shows the novel arrangement of the electrodes 1 and 2 of the nanogap sensor 100, which are manufactured from two different materials, like e.g. a carbon-based material, e.g. doped diamond, on the one hand and silicon on the other hand. The two electrodes 1 and 2 are separated from one another by an isolator 12, which here is recessed bilaterally, so that a gap with a size of a few 10 nm is formed between the electrodes 1 and 2. Such a recess is not necessarily required, and no gap must exist. A further possibility would be a freely floating construction without a supporting isolator in-between.

An at least partially longitudinally oriented probe molecule (affinity molecule A or 3, respectively) is directly or via a linker bound to electrode 1 with at least one of its peripheral ends (sensor-binding ends), and thus immobilized there, while at least one of its free (=peripheral) ends protrudes from electrode 1.

In the same manner, an—at least partially-longitudinally oriented probe molecule B or 4, respectively, which is directly or indirectly bound to electrode 2 with one of its peripheral ends and thus immobilized there, freely protrudes from electrode 2 with at least one of its free ends. To the two free ends of the two probe molecules A,3 and B,4, the analyte molecule C or 5, respectively, is bound with two of its respective ends, the analyte molecule originally stemming from the fluid medium Mf and deposited on and bound to the two probe molecules A,3 and B,4, wherein in total a bridge Bm is formed, bridging the nm gap and simultaneously connecting electrodes 1 and 2 with one another.

Thus, a transition has taken place, from a condition with two probe molecules A,3 and B,4 protruding from electrodes 1 and 2 to a bridge Bm including the analyte molecule C,5 and interconnecting the electrodes, which results in a metrologically detectable alteration of the alternating current impedance and enables an inference on the presence and possibly also the quantity of the analyte molecule C5 in the fluid medium.

FIG. 1b shows—otherwise using the same reference numbers—the formation of the bridge Bm between electrodes 1 and 2 more clearly.

Probe A,3 is bound to electrode 1 with its sensor-bound binding site a31 and probe B,4 is bound to electrode 2 with its sensor-bound binding site b41. The affinity binding sites a32 and b42 of the two probe molecules A,3 and B,4 respectively, have formed one or several bond(s) with the two substantially terminal or exposed, respectively, binding sites c53 and c54 of the analyte molecule C,5 and in total form the bridge Bm, which interconnects the two electrodes 1 and 2 across the nm gap.

FIG. 2 shows—otherwise using the same reference numbers—an inverse process. A “pre-bound situation” exists, with an existing bridge Bm between electrode 1 and 2, which has an auxiliary molecule D,6, e.g. a piece of DNA strand, as a bridge component. D,6 is not necessarily an analyte molecule.

In the fluid medium exists a complementary analyte molecule C,5 attachable and bondable to the piece of strand or the auxiliary molecule D,6, respectively, which attaches itself to the auxiliary molecule D,6, and the bonds thereof to the affinity binding sites of the probe molecules A,3 B,4 are dissolved, whereby the bridge Bm is destroyed, which again results in a measurable impedance modification, which enables inferences on the presence and possibly also on the quantity of the analyte molecule C,5.

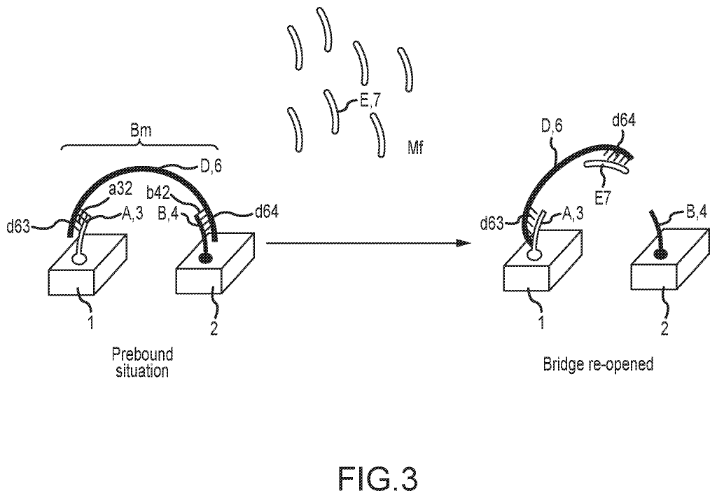

FIG. 3 shows—otherwise using the same reference numbers-a process generally similar to FIG. 2. Here, molecules E,7 are present in the fluid medium Mf, which bind to only one of the two peripheral binding sites d63,d64 of the auxiliary molecule D,6, namely d64, whereat the binding power is higher than the bond d64-b42 with the probe molecule B,4.

The just stated bond is dissolved and the molecule E,7 binds to the binding site d64 of the auxiliary molecule D,6, whereby the original bridge Bm no longer exists and an impedance modification can be observed again.

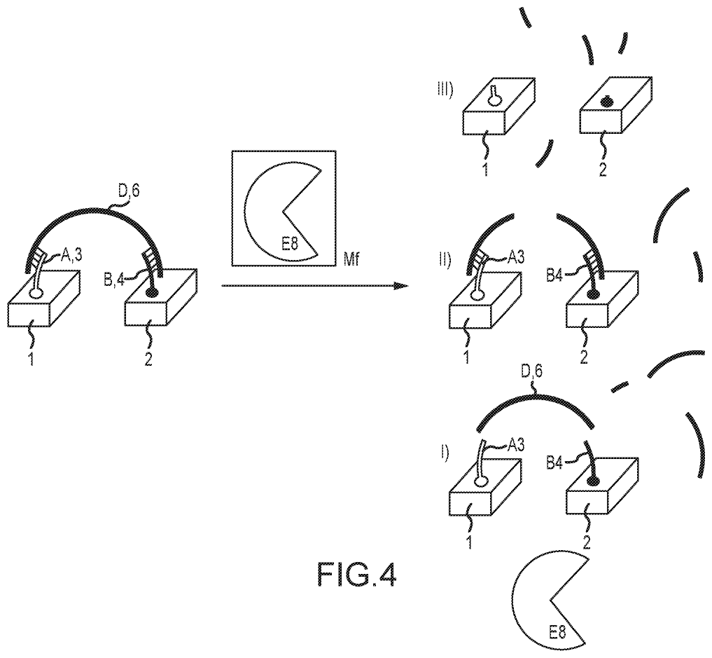

FIG. 4 shows—otherwise using the same reference numbers-a bridge Bm formed with the probe molecules A,3 and B4 sensor-bound to the two electrodes 1 and 2 and bound to the exposed binding sites of an auxiliary molecule D,6 with their affinity bonds, which e.g. is destroyed by an enzyme E,8 in three different ways, namely I) by detachment of the auxiliary molecule D,6 from the probe molecules A,3, B,4 by removal of the double-strand areas, II) by destruction of the auxiliary molecule D,6 in the single-strand area, or III) by destruction of the probe molecules A,3, B,4 and the auxiliary molecule D,6 involved in the original bridge Bm. With the destruction of the bridge Bm, there is a modification of the impedance, and thus the presence, and via measurement of the kinetic effects, also the concentration of enzyme E,8 in the fluid medium Mf can be concluded.

Additional Aspects

1. A method for identifying organic and biochemical substances, in particular molecules, molecule sequences, molecule parts or the like, and for determining their quantity or concentration, respectively, in a fluid, i.e. liquid or gaseous, medium, wherein a nanogap sensor is used, which comprises at least two electrodes, characterized in that:
a nanogap sensor (100) is used, the at least two electrodes (1,2) of which are separated from one another by an electrically isolating layer (12) or by a non-material gap (12), said electrodes being formed of materials being different from one another, electrically conductive and/or principally semi-conductive, however, having a relatively high conductivity with regard to the semiconductor characteristic,
on the surface of said first electrode (1) of said sensor (100), a first affinity or probe molecule A(3), respectively—preferably equipped with an at least partial longitudinal orientation—with a sensor-binding area (a31) is specifically or individually, respectively, sensor-bound to the material of said first electrode (1) at one of its ends or in the proximity of one of its ends, and is immobilized there, wherein the free residue of said first probe molecule A(3) has at least one, preferably, however, several free binding or bondable, respectively, group(s), molecule sequence(s) or the like representing affinity binding sites (a32), which have at least certain specificity for bonding to a sought substance, in particular to an analyte or analyte molecule C(S), respectively, or auxiliary molecule D(6),
on the surface of said second electrode (2) of said sensor (100), a second affinity or probe molecule B(4), respectively—preferably likewise equipped with an at least partial longitudinal orientation—differing in relation to said first affinity or probe molecule A(3), with a binding area (b41) is specifically or individually, respectively, sensor-bound to the material of said second electrode (2), the material of which differs from the material of said first electrode (1), at one of its ends or in the proximity of one of its ends, and is immobilized there, wherein the free residue of this second probe molecule B(4) as an affinity binding site (b42) has at least one, preferably, however, several free binding or bondable, respectively, group(s), molecule sequence(s) or the like, likewise with at least certain specificity for bonding to a sought substance, in particular to an analyte or analyte molecule C(5), respectively, or auxiliary molecule D(6),
from a fluid medium (Mf) to be checked—usually containing various molecules, molecule sections or parts, respectively, molecule sequences or the like and flowing around said electrodes (1,2) and said isolator or gap (12), respectively, there between—an analyte molecule C(5) or auxiliary molecule D(6) sought as such, in particular to be determined in its quantity and/or concentration, formed by a known molecule, such molecule section or part, respectively, such molecule sequence or the like, essentially of any shape, respectively with at least two binding sites (c53, c54; d63, d64) at a distance from one another for said sensor-bound, immobilized probe molecules A(3) and B(4) or for their mobile free ends with free affinity binding sites (a32) and (a42), respectively, selectively passes out of the fluid medium (Mf) to be analyzed, in which it is contained, and using said exposed binding sites (c53, c54; d63, d64) respectively arranged at its exposed points, as in particular at the various ends or in their proximity, respectively, forms a bond with particularly binding or bondable, respectively, groups, molecule sequences or the like with the binding or bondable, respectively, groups, molecule sequences or the like representing the affinity binding sites (a32, b42), respectively, at the free mobile ends of said first and second affinity or probe molecules A(3) and B(4), respectively, specifically sensor-bound to said electrodes (1,2) of different materials with their respectively other peripheral ends or terminal areas, respectively, via the sensor binding sites (a31, a41) there, or immobilized there, respectively—forming a bridge molecule or bridge (Bm), respectively, ultimately connecting said two electrodes (1,2) of different materials with one another, overall formed by said probe molecules A(3) and B(4) and said analyte molecule C(5) or auxiliary molecule D(6)—respectively bridging said isolator layer or gap (12), respectively, between said two electrodes (1,2) of different materials, or
said auxiliary molecule D(6), contained in existing bridges, bound to said two probe molecules A(3) and B(4)—respectively sensor-bound with their sensor binding sites (a31,b41) to said electrodes (1,2) of different materials themselves—via their affinity binding sites (a32,b32), is separated from at least one of said probe molecules A(3) and B(4) bound to said electrodes (1,2) of different materials by the interaction of an analyte molecule E(7) contained in the fluid medium (Mf), which is capable of binding to at least one binding site (d63,d64) of said auxiliary molecule D(6), which bond is stronger than at least one of the binding groups (a32d63,b42d64) between said auxiliary molecule D(6) and at least one of said two probe molecules A(3), B(4), i.e. a previously existing bridge (Bm) is dissolved, and
on the basis of the modification of impedance or of the frequency spectrum of an alternating current applied to said two electrodes (1,2) of different materials, respectively, occurring during the bridge formation or the bridge dissolution process, the presence, the quantity or concentration, respectively, of the sought molecule is determined, wherein in case of a bridge formation, the absence of the sought analyte molecule C(S) at the electrodes existing before the bridge molecule formation and the presence of said sought analyte molecule C(S) at the electrodes present following the bridge formation, or in case of the bridge dissolution, a selective connection of said analyte molecule C(S) to component(s) of a bridge pre-manufactured using an auxiliary molecule D(6), results in the modification, no matter, whether said analyte molecule C(S) has formed a direct or indirect connection with the sensor surface or such connection has been dissolved following the reaction.

2. The method according to 1, characterized in that a dissolution of a bridge (Bm) formed with said probe molecules A(3) and B(4) bound to each of said two electrodes (1,2) of different materials on the one hand, and with said auxiliary molecule D(6) bilaterally bound with these via their affinity binding sites (a32, b42), in particular with a DNA sequence strand, on the other hand, is executed by supplying an analyte molecule C(S), in particular a piece of complementary DNA sequence, essentially attachable to said auxiliary molecule D(6) and highly bondable to the same, using the fluid medium (Mf, which analyte molecule D(S) binds to said auxiliary molecule D(6), in particular to a respective DNA sequence strand, and forming a doublemolecule, in particular a DNA double-strand, which ultimately migrates into the fluid medium, said two affinity bonds (d63a32,d64b42) to said two probe molecules A(3) and B(4), sensor-bound to said electrodes (1,2) of different materials, are dissolved (FIG. 2).

3. The method according to 1 or 2, characterized in that a dissolution of a bridge (Bm) formed with said probe molecules A(3) and B(4) sensor-bound to each of said two electrodes (1,2) of different materials on the one hand, and with said auxiliary molecule D(6) bound with these via their affinity binding sites (a32, b42), in particular with a DNA sequence strand, on the other hand, is executed using said analyte molecule E(7) present in the fluid medium (Mf), which has a molecule group attachable and bondable to only one of the two exposed binding sites or groups (c53,c54), respectively, of said auxiliary molecule D(6) initially bound to said affinity binding sites (a32,b42) or binding groups, respectively, of said two immobilized probe molecules A(3) and B(4) sensor-bound to said two electrodes of different material, dissolving only one of said two affinity bonds (d63a32, d64b42) with one of said two sensor-bound probe molecules A(3) and B(4), and thus dissolving the bridge (Bm), binding to one of said thus released two exposed binding sites or binding groups (d63,d64) of said auxiliary molecule D(6), respectively (FIG. 3).

4. The method according to any of 1 to 3, characterized in that a nanogap sensor (100) is used, in which the distance or the thickness, respectively, of said isolating layer or gap (12), respectively, between said two electrodes (1,2) of different materials lies in the magnitude of up to 500 nm, preferably of up to 200 nm, in particular in the range from 20 to 70 nm.

5. The method according to any of 1 to 4, characterized in that a nanogap sensor (100) is used, in which the distance between said two electrodes (1,2) of different materials is formed by a layer of a solid or liquid dielectric material, for example of inorganic isolator materials from the fields of microelectronics or field-effect transistor applications, in particular oxides, nitrides and/or chalcogenides, for example silicon oxide, silicon nitride, aluminum oxide, zirconium oxide, silicone nitride, tantalum pentoxide or thin-layer films of various origins, like in particular Langmuir-Blodgett (LB) films, polyelectrolyte multi-layers and self-organized monolayers of different materials and material combinations, as well as diverse polymers, which compared to the lower conductivity of the two measuring electrodes have a conductivity, which is lower by at least three powers of ten, like for example Kapton®, Nafion® or others, which are known to the person skilled in the art, particularly preferred isolation materials routinely used today already or in future, producible in reproducible layer thicknesses within the scope of micro-system technology, like in particular Si-oxide and Si-nitride. In case of completely undercut nano-belts, the isolating layer may likewise be identical with the electrolyte.

6. The method according to any of 1 to 5, characterized in that a nanogap sensor (100) is used, said two electrodes (1,2) of different materials of which may in their property-determining material respectively be formed of a combination of any two of the materials listed in the following, wherein the combinations may be formed within or outside the material groups: metals, like for example gold, platinum, silver, mercury; doped semiconductors, like for example silicon and germanium; Hl-V or M-V1, respectively; semiconductors, like for example GaAs, CdS, CdSe, CdTe; carbonaceous layers, like for example graphite, fullerenes, nano-tubes, diamond-like carbon, diamond in various versions, e.g. as mono-crystal, micro-crystalline, preferably, however, nano- or ultra-nano-crystalline, as well as material combinations, alloys, including doping, or further electrode materials known per se, like in particular gallium nitride, SiC (silicon carbide), AIN (aluminum nitride), ATO or ITO, wherein a combination of two highly doped non-metals is particularly preferred, in this connection particularly preferred a combination of highly doped, almost metallically conductive silicon with highly doped diamond, in particular UNCD (ultra-nano-crystalline diamond).

7. Use of the method according to any of 1 to 6 for the verification of a biochemical process, in which not said affinity bonds (d63a32 or d64b42) of said auxiliary molecule D(6) to said two probe molecules A(3), B(4) for bridging said two electrodes (1,2) are dissolved, but said bridge-forming auxiliary molecule D(6) is destroyed at at least one random point, e.g. for the detection of DNases or proteolytically effective enzymes F(8) (FIG. 4).

8. The use of the method according to any of 1 to 6 for the verification of a biochemical process, in which different biomolecules serve as at least one of said affinity or probe molecules A(3) and B(4), respectively, like e.g. antibodies, which via said or using said, respectively, analyte molecule C(S) cause the formation of a bridge, or nucleic acid sequences, which by means of hybridization cause bridging of said electrodes (1,2) of different materials, wherein the possibility of linker sequences and spacers, which guarantee an increased mobility of said probe molecules A(3) and B(4) compared to the surfaces of said electrodes (1,2) of different materials, is included.

9. The use of the method according to any of 1 to 6 for the verification of a biochemical process, in which artificially generated analogues of biomolecules, like e.g. PNAs and LNAs, are used as said affinity or probe molecules A(3) and B(4).

10. The use of the method according to any of 1 to 6 for the verification of a biochemical process, in which by means of a continuous chemical reaction, like e.g. by means of polymerase chain reaction (PCR), a band between said two electrodes (1,2) of different materials or between said two probe molecules A(3) and B(4) unilaterally sensor-bound to these, respectively, is linked or destroyed, which is used for detection.

### QUOTATIONS

### Patents

US 2002/0172963 A1: “DNA bridged carbon nanotube arrays”, Inventors: Kelley; Fourkas; Naughton; Ren (all US)
US 2002/0022223 A1: “High resolution DNA detection methods and devices”, Inventor: Connolly/US
US 2004/012161 A1: “Method for quantitative detection of nucleic acid molecules”, Inventor: Connolly/US
US 2005/0287589 A1: “High resolution DNA detection methods and devices”, Inventor: Connolly/US
US 2006/0019273 AV: “Detection card for analyzing a sample for a target nucleic acid molecule, and uses thereof”, Inventors: Connolly; Hainon; Murante; Grece; Tiller (all US)

### Articles

Braun E., Eichen Y., Sivan U., Ben-Yoseph G. Nature. 1998 Feb. 19; 391(6669):775-8
Chen F., Qing Q., Ren L., Wu Z. and Liu Z. APPLIED PHYSICS LETTERS 86, 123105 s2005d
Hashioka S., Saito M., Tamiya E., Matsumura H. Appl. Phys. Lett. 85 (2004) 687
He H. X., Boussaad S., Xu B. Q., Li C. Z., Tao N. J. Journal of Electroanalytical Chemistry 522 (2002) 167-172
Hwang J. S., Kong K. J., Ahn D., Lee G., Ahn D. J., Hwang S. W. Appl. Phys. Lett. 81 (2002)1134
Iqbal S. M., Balasundarama G., Ghosh S., Bergstrom D. E., Bashir R. APPLIED PHYSICS LETTERS 86, 153901 s2005d
Liu B., Xiang J., Tian J.-H., Zhong C., Mao B.-W., Yang F.-Z., Chen Z.-B, Wu S.-T., Tian Z.-Q. Electrochimica Acta 50 (2005) 3041-3047
Qing Q., Chen F., Li P., Tang W., Wu Z., Liu Z. Angew. Chem. Int. Ed. 2005, 44, 7771-7775
Reichert J., Ochs R., Beckmann D., Weber H. B., Mayor M., H. v. Lohneysen VOLUME 88, NUMBER 17 PHYSICAL REVIEW LETTERS 29 Apr. 2002
Reed M. A., Zhou C., Muller C. J., Burgin T. P., Tour J. M. 252-254 SCIENCE VOL. 278 10 Oct. 1997
Sambrook et al. “Molecular cloning”, 3^{rd} Ed., CSHL Press

### Poster

Roppert, K., Heer, R., Kast, M., Stepper, C., Koeck, A., Brueckl, H.: “A new approach for an interdigitated electrodes DNA-sensor”, presented at the Bioelectrochemistry 2005 in Coimbra.

1. A method of detecting a nucleic acid analyte, the method comprising:
exposing a closed circuit to the nucleic acid analyte in a fluid medium, the closed circuit comprising:
a first electrode;
a second electrode spaced apart from the first electrode by a nanogap:
a first affinity probe bonded to the first electrode;
a second affinity probe bonded to the second electrode;
a single-stranded DNA oligonucleotide bridge molecule bonded at each end to the first and second affinity probes, bridging the nanogap;
binding the nucleic acid analyte to the first affinity probe while correspondingly dissolving the bond between the first affinity probe and an end of the single-stranded DNA oligonucleotide bridge molecule, thereby opening the closed circuit; and
observing a resulting change in impedance from the opening of the closed circuit, the change in impedance indicating the presence of the nucleic acid analyte in the fluid medium.

2. The method of claim 1, wherein the first electrode comprises diamond and the second electrode comprises silicon.

3. The method of claim 2, wherein the bond between the diamond electrode and the first affinity probe comprises a bifunctional crosslink between an amino group of a phenylamino moiety immobilized on the diamond electrode and an amino group present on the first affinity probe, the bifunctional cross-link comprising the two amino groups and a phenylene di-isothiocyanate cross-linker.

4. The method of claim 1, wherein the first electrode comprises silicon and the second electrode comprises diamond.

5. The method of claim 4, wherein the bond between the diamond electrode and the second affinity probe comprises a bifunctional crosslink between an amino group of a phenylamino moiety immobilized on the diamond electrode and an amino group present on the second affinity probe, the bifunctional cross-link comprising the two amino groups and a phenylene di-isothiocyanate cross-linker.

6. The method of claim 1, wherein the closed circuit further comprises at least one helper oligonucleotide bonded to the single-stranded DNA oligonucleotide bridge molecule initially bridging the nanogap in the closed circuit.

7. The method of claim 1, wherein the nanogap measures from about 20 nm to about 70 nm.

8. The method of claim 1, wherein the single-stranded DNA oligonucleotide bridge molecule initially bridging the nanogap in the closed circuit comprises at least one point mutation resulting in weaker binding between the single-stranded DNA oligonucleotide bridge molecule and the first affinity probe relative to binding between the analyte and the first affinity probe.

9. The method of claim 1, wherein the closed circuit further comprises an external reference electrode.

10. The method of claim 1, wherein the first and second electrodes are electrically connected to a measuring device.

11. The method of claim 10, wherein the measuring device is capable of measuring DC voltage offset impedance.

12. The method of claim 1, further comprising equilibrating the electrodes with a buffer that does not comprise the analyte, prior to exposing the closed circuit to the analyte in the fluid medium.

13. A method of detecting a nucleic acid analyte, the method comprising:
exposing a closed circuit to the analyte in a fluid medium, the closed circuit comprising:
a first electrode;
a second electrode spaced apart from the first electrode by a nanogap, wherein the first electrode comprises silicon and the second electrode comprises diamond and the bond between the diamond electrode and the second affinity probe comprises a bifunctional crosslink between an amino group of a phenylamino moiety immobilized on the diamond electrode and an amino group present on the second affinity probe, the bifunctional cross-link comprising the two amino groups and a phenylene di-isothiocyanate cross-linker;
a first affinity probe bonded to the first electrode;
a second affinity probe bonded to the second electrode; and
a single-stranded DNA oligonucleotide bridge molecule bonded at each end to the first and second affinity probes, bridging the nanogap;
binding the nucleic acid analyte to the first affinity probe while correspondingly dissolving the bond between the first affinity probe and an end of the single-stranded DNA oligonucleotide bridge molecule, thereby opening the closed circuit; and
observing a resulting change in impedance from the opening of the closed circuit, the change in impedance indicating the presence of the nucleic acid analyte in the fluid medium.

14. The method of claim 1, further comprising an auxiliary molecule that binds to the single-stranded DNA oligonucleotide bridge molecule to the first affinity probe or the second affinity probe to dissolve the bond between the single-stranded DNA oligonucleotide bridge molecule and the first or second affinity probe, wherein the auxiliary molecule is destroyed at at least one random point.

15. The method of claim 13, wherein the method is used for the detection of DNases or proteolytically effective enzymes.

16. The method of claim 1, wherein the single-stranded DNA oligonucleotide bridge molecule comprises one or more point mutation.

17. A method of detecting a nucleic acid analyte, the method comprising:
exposing a closed circuit to the nucleic acid analyte in a fluid medium, the closed circuit comprising:
a first electrode;
a second electrode spaced apart from the first electrode by a nanogap;
a first affinity probe bonded to the first electrode;
a second affinity probe bonded to the second electrode;
a single-stranded DNA oligonucleotide bridge molecule bonded at each end to the first and second affinity probes, bridging the nanogap, wherein the single-stranded DNA oligonucleotide bridge molecule initially bridging the nanogap in the closed circuit comprises at least one point mutation resulting in weaker binding between the single-stranded DNA oligonucleotide bridge molecule and the first affinity probe relative to binding between the analyte and the first affinity probe;
binding the nucleic acid analyte to the first affinity probe while correspondingly dissolving the bond between the first affinity probe and an end of the single-stranded DNA oligonucleotide bridge molecule, thereby opening the closed circuit; and
observing a resulting change in impedance from the opening of the closed circuit, the change in impedance indicating the presence of at least one point mutation in the nucleic acid analyte in the fluid medium.
